(19) United States
(12) Patent Application Publication
(10) Pub. No.: US 2010/0010077 A1
(43) Pub. Date: Jan. 14, 2010

(54) PROCESS FOR THE ADDITIVATION OF WINE

(75) Inventors: Luis Manuel Da Costa Cabral E Gil, S. Domingos de Rana (PT); Carlos Rodrigues Pereira, Amadora (PT)

Correspondence Address: SCULLY SCOTT MURPHY & PRESSER, PC, 400 GARDEN CITY PLAZA, SUITE 300, GARDEN CITY, NY, 11530, US

(73) Assignee: Instituto Nacional de Engenharia Tecnologia E Inov, Lisboa (PT)

(21) Appl. No.: 12/374,714

(22) PCT Filed: Jul. 23, 2007
(86) PCT No.: PCT/PT07/00032

§ 371 Date: Jan. 22, 2009

(30) Foreign Application Priority Data
Jul. 24, 2006 (PT) 103535

(51) Int. Cl. A61K 31/352

(52) U.S. Cl. 514/456

(57) ABSTRACT

The present invention relates to a process for the additivation of wine, more specifically for the formation of compounds that are beneficial to health. The process is based on the contact of natural materials, namely cork, with wine, under conditions where certain compounds of the said materials react with other compounds present in the wine in order to form therein compounds with properties that are beneficial to the health of the consumer, namely a potent anti-cancerous agent called acutissimin-A. Additionally, the contact of the wine with the material also promotes the passage and interaction of compounds which will organoleptically influence the wine. The additivation process involves the contact with cork usually in fines particles, either at the final stage of vinification before bottling, during bottling or immediately before consumption, through different retention systems (filters, cartridges, etc.).

It has been known for a long time that wine in general, and red wine in particular, is beneficial to health if consumed in moderation. Studies which have been carried out in the last few years state that this characteristic is due to several components, some of which have effect at cardiovascular level and others having anti-cancerous properties, etc. Antioxidants such as quercetin, resveratrol and catechin are able to inhibit the development of certain cancers. There is an extensive bibliography in this respect, of which a good summary can be found in the Annals of the New York Academy of Sciences, vol. 957, 2002, “Alcohol and Wine in Health and Disease”, Editors Dipak, K. Das & Fulvio Ursini.

The ageing of wine in oak (either in barrels or with chips) is well known and confers certain organoleptic characteristics on the wine, the interaction between wine and cork being sometimes also mentioned although there is no scientific evidence of this to date.

With regard to the additivation of wine, a patent search has shown the existence of the patent RU 2155216, which relates to an alcoholic drink being enriched with compounds having antioxidant properties, but not according to the process proposed by this invention, and also the patent WO 9963048, which discloses a process for the manufacture of antioxidant-enriched beverages by maceration with tannin-rich vegetable material.

### BACKGROUND OF THE INVENTION

Studies conducted by various researchers have demonstrated that some components of cork with interest as regards the development of wine may migrate thereto when in contact with the wine, namely volatile organic compounds that account for certain odours and flavours. Among these compounds of interest are, for instance, vanillin and ellagitannins which, when complexed with anthocyanins, affect the astringency and other characteristics of wine. Regarding the polyphenols capable of migrating from cork to wine, reference can be made to the article published by Varea S. et al., “Polyphenols susceptible to migrate from cork stoppers to wine”, Eur. Food. Res. Technol., 213: 56-61, 2001.

In another study, it was concluded that when wine was contacted with oak wood (in ageing processes using oak barrels), the ellagitannins present in wood, particularly one of them called vescalagin, reacted with the catechins contained in the wine, thus producing acutissimin-A, an extremely potent anti-tumour agent (see Quideau, S. et al., “DNA topoisomerase inhibitor acutissimin-A and other Flavano-ellagitannins in red wine”, Angew. Chem. Int. Ed., 42: 6012-6014, 2003).

The inventors of this invention were able to demonstrate, surprisingly, that the same happened when wine was made to contact with cork. Although cork also contains vescalagin in its composition, as stated by Varea (2001) above, it was not possible to draw such a conclusion because there are significant differences between the composition of cork and wood, meaning that there might be inappropriate proportions between the constituents, interferences with other constituents, parallel reactions, etc., susceptible of hindering the formation of acutissimin-A.

The cork used in this invention comes from a very specific kind of tree, the cork-oak (Quercus suber L.), which is subject to successive extractions with the aim of providing a material from its bark, reproduction cork, having completely different properties compared to wood. Thus, cork is a specific material, whose chemical and morphological constitution is very distinct from that of wood. According, for example, to Guillemonat, A., “Progrès récents dans l'étude de la constitution chimique du liège”, Ann. Fac. Sc. Marseille, 30: 43-54, 1960 and Pereira, H., “Chemical composition and variability of cork from Quercus suber L.”, Wood Sci. Technol., 22(3):211-218, 1988, it may be considered that the chemical composition of cork is, typically, as follows:
46% suberin (main constituent)
25% lignin
12% cellulose and other polysaccharides
6% tannins
6% ceroids
5% ashes and other compounds

By way of comparison, the typical chemical composition of wood, for example Fengel D., Wegener D., “Wood: chemistry, ultrastructure, reactions”, Walter de Gruyter, Berlin, 1989, is:
60% cellulose (main component)
25% lignin
15% other compounds

This difference in composition makes cork a specific tissue which is fundamentally distinct from wood.

The contents of each of the references mentioned above are herein incorporated in their entirety.

### DETAILED DESCRIPTION OF THE INVENTION

The present invention relates to a process for the formation of compounds in wine which are beneficial to health, by contacting the wine with natural products containing vescalagin. Preferably, the vescalagin-containing natural product is the cork from Quercus suber L. More specifically, this invention relates to the contacting of wine with cork in such conditions that the vescalagin of the cork is reacted with the catechins existing in the wine, in order to produce therein acutissimin-A. The reaction conditions (temperature, contact time, cork granulometry, cork-wine ratio, etc.) may vary, but are preferably room temperature, a period of contact of 30 minutes or more and a granulometry of 1-5 mm. The weight/volume (w/v) or volume/volume (v/v) ratio of cork/wine can vary, but it is preferably from 1/100 to 3/100 (g/ml) or from 20/100 to 40/100 (ml/ml) respectively.

The process for the additivation of wine involves contact with cork, usually in finely divided particles or in the form of chips, either added directly to the wine and then separated by filtration before or during bottling, or prior to consumption by means of retention systems which contain the cork and can be easily introduced into and removed from the wine containers. In a preferred embodiment, the said systems are cartridges, porous capsules, small perforated metal frameworks or net systems and other similar systems. Also in a preferred embodiment, the cork particles used in these systems must be sterilized, before or after their closure into the systems, for example by gamma ray or electron beam irradiation, or even with the objective of avoiding a musty taste and flavour in the wine, according to the method disclosed in the Portuguese patent application PT 103006 owned by the same inventors.

It was also discovered that, when contacting with cork, the wine acquires some of its volatile and extractable compounds which alter the wine, causing it to slightly change in colour, namely in the case of white wines, and giving it a taste which in wine jargon is referred to as a rounder, less acidic and woody taste, these attributes being considered as positive by most consumers.

This process has the advantage of being very simple and cheap, also allowing the formation of beneficial compounds in wine with very short periods of contact, which can be used as a complement in the wine-making process or by the consumer himself.

The process will be better understood by means of the following examples, which are intended merely for illustrative and not restrictive purposes.

### Example 1

### a. Samples Used

Red wine; Year 2003; Grape varieties—Touriga Franca and Touriga Nacional; Trademark—Quinta do Cardo; Producer—Companhia das Quintas (no holding time in wood, namely oak wood).

Cork stoppers supplied by the company Juvenal Ferreira da Silva S.A., size 44×24 mm, 3^{rd} category, with Suncl washing and without any surface treatment.

Acutissimin standard given by Quideau (Quideau, S., Jourdes, M., Lefeuvre, D., Montaudon, D., Saucier, C., Glories, Y., Pardon, P. and Pourquier, P., 2005, “The Chemistry of Wine Polyphenolic C-Glycosidic Ellagitannins Targeting Human Topoisomerase II”, Chem. Eur. J. 11, 6503-6513).

### b. Extraction/Reaction Conditions

The assays were performed in 250 ml Erlenmeyers with 100 ml wine in contact with a material volume (cork granulate) of approximately 30 ml corresponding to 2.5 g of cork. The cork was previously ground in a Retsch mill to a size of 1.5 mm or less. The flasks in which the wine and the source of vescalagin were contacted were maintained under horizontal stirring during the trial periods at a room temperature of about 21° C. All the assays were carried out in duplicate.

### c. Analytic Conditions

The determination of the presence of acutissimin-A in red wine samples in contact with cork was carried out by ESI (electrospray ionization)—IDT (ion trap detector)—MS/MS (LC-MS). The experimental method used was the one disclosed in literature with slight changes (Quideau, S., Jourdes, M., Lefeuvre, D., Montaudon, D., Saucier, C., Glories, Y., Pardon, P. and Pourquier, P., 2005, “The Chemistry of Wine Polyphenolic C-Glycosidic Ellagitannins Targeting Human Topoisomerase II”, Chem. Eur. J. 11, 6503-6513), since the abovementioned method is aimed at separating/identifying polyphenols and the interest of this work is focused only on determining the presence of acutissimin-A.

A high performance liquid chromatograph hyphenated with a mass spectrometer was used, with the following specifications: a Waters high performance liquid chromatograph (model Alliance 2695), equipped with a quaternary gradient pump, an automatic injector, a column oven, a photodiode detector (model 996), hyphenated with a Bruker mass spectrometer (model Esquire 3000) with electrospray ionization (ESI source) and an ion trap detector (ITD) and data acquisition and treatment systems of Esquire and HyStar.

The specifications of the liquid phase chromatography were as follows:
Column: Hypersil ODS column, 250-4 mm, 5 μm particle size, Hypersil, Thermo
Mobile phase: Gradient 3, wherein:
A=H₂O with 0.065% TFA (v/v)
B=MeOH with 0.065% TFA (v/v)

TABLE 1

| Time (min) | Flow (ml/min) | % A | % B |
|---|---|---|---|
| | 1.0 | 100 | 0 |
| 10 | 1.0 | 80 | 20 |
| 30 | 1.0 | 60 | 40 |
| 31 | 1.0 | 100 | 0 |
| 36 | 1.0 | 100 | 0 |

Detection: UV at 240 nm
Injection Vol.: 10 and 109 μl
Mobile Phase Flow: 1.0 ml
Rt_{Acutissimin-A}: ≈11.8 min.
   The mass spectrometry specifications were as follows:
ESI source conditions:
   Nebulization gas pressure (N₂): 50 psi
   Drying gas flow (N₂): 10 L/min
   Drying gas temperature: 365° C.
Acquisition parameters:
   Mode of ionization: negative ions
   Ion trap conditions:
      mass screening range: 850-1250 m/z
      accumulation time: 100 ms
      maximum number of accumulated ions: 100000
   Voltage applied to the cone (Skim 1): −57.2 V
   Voltage applied to the capillary: +4000 V
Optimization parameters:
   Target mass: 1205 m/z
   Compound stability: 70%
   Ion trapping level: 100%
Fragmentation parameters:
   MS² fragmentation sequence studied: 1205→915
   Precursor ion isolation width: 1 m/z
   Precursor ion fragmentation amplitude: 1 V

The first step of identification by mass spectrometry comprised the direct insertion of the acutissimin-A standard into the analytic system, followed by the confirmation of the spectrum obtained by injection using HPLC. The analysis by LC-ESI-MS of the standard showed that it was rather impure, co-elution with other compounds having even occurred. An MS-MS ion fragmentation study was also carried out at m/z=1205, by direct injection, thus obtaining the daughter ion with m/z=915. The acutissimin-A spectrum has 2 characteristic signals (the more intense ones) (FIG. 1), with ESI ionization in negative mode, at m/z 1205 (base signal), corresponding to the molecular ion (M-H), and at m/z 915.

In order to confirm whether the compound with a signal at m/z 1205 could be explained by the presence of acutissimin-A, the said signal was subjected to MS-MS fragmentation using a fragmentation amplitude of 1.00 V. This test was conclusive as regards the presence of the desired compound, since the daughter ion of m/z=915 was obtained, as with the standard. The same method was applied to the red wine sample which had not been contacted with cork (blank assay) and the result was the absence of the compound of interest, i.e. acutissimin-A. The next step consisted of concentrating the acutissimin-A contained in 50 ml of red wine, using SPE RP-18, 500 mg. The column was preconditioned with 3 ml of ethanol and the sample was then applied. The analyte was eluted with 3 ml H₂O and 1 ml ethanol. The analysis of the red wine samples thus prepared, following a 24 hour contact with cork, revealed the presence of acutissimin-A.

### Example 2

In the second example, the assays were carried out based on contact times of 30 min, 2 h 30 min and 6 days of the red wine described in Example 1 with cork granulate. The analyses were conducted according to the procedures disclosed in Example 1.

The following table shows the results obtained in these assays (Table 2):

TABLE 2

| | | | | Absolute area | | |
|---|---|---|---|---|---|---|
| Sample | | Contact | | Ref. signal | Acutissimin-A | Relative area (%) |
| Reference | | Time | | rₜ(1) = 9 min | rₜ(2) = 12 min | [Area (2)/Area (1)]*100 |
| Cork | concentrated | 30 | min | 94.59 | 5.42 | 5.73 |
| | as is | 30 | min | 89.94 | 10.06 | 11.19 |
| | concentrated | 150 | min | 94.14 | 5.87 | 6.24 |
| | as is | 150 | min | 88.23 | 11.77 | 13.34 |
| | concentrated | 6 | days | 91.47 | 8.53 | 9.33 |
| | as is | 6 | days | 90.95 | 9.06 | 9.96 |

| | | |
|---|---|---|
| Wine sample | — | — |
| without contact¹ | | |

¹Process blank

As may be seen from the analysis results in Table 2, acutissimin-A is detected in all the samples in which the wine has contact with the cork. In the blank sample, as expected, acutissimin-A was not detected.

FIGS. 2, 3 and 4 show the spectra obtained from the injection into the liquid phase chromatograph of the as-is samples of red wine in contact with cork granulate, for 30 min, 150 min and 6 days respectively. In all the three cases, the existence of the signal corresponding to acutissimin-A is marked with the number 2.

### Example 3

Assays were conducted using the same procedure as in example 1, except that the contact between wine and cork was carried out with the cork placed inside 100×50 mm cotton cartridges. The assays were carried out using white wine with the following characteristics:

Year 2004; Grape variety—Arinto; Trademark—Prova Régia; Producer—Companhia das Quintas (no holding time in wood, namely oak wood).

The red wine used was the same as in example 1. The assays were carried out for short contact times (cork/wine)—30 and 150 min.

The results are summarized in Table 3. The detection of acutissimin-A was confirmed by the presence of the signal m/z at 915 by EIC. The signal intensity was also measured.

TABLE 3

| | Signal | Signal | | |
|---|---|---|---|---|
| | m/z = 915 | Intensity | Rt (signal | Average |
| SAMPLES | by EIC | m/z = 915 | of m/z = 915) | Intensities |
| Red 30 min 1^{st} injection | No | 7 | 11.7 | 7.0 |
| Red 30 min 2^{nd} injection | Yes | 7 | 11.6 | |
| White 30 min 1^{st} injection | Yes | 12 | 11.7 | 19.5 |
| White 30 min 2^{nd} injection | Yes | 27 | 11.7 | |
| Red 150 min 1^{st} injection | Yes | 6 | 11.6 | 7.5 |
| Red 150 min 2^{nd} injection | Yes | 9 | 11.7 | |
| White 150 min 1^{st} injection | Yes | 10 | 11.8 | 9.0 |
| White 150 min 2^{nd} injection | Yes | 8 | 11.7 | |

Rₜ—Retention time

It may be concluded from an analysis of Table 3 that acutissimin-A is also formed for short contact times even if such contact is not made in a free direct way.

Another conclusion is that, as in the case of red wine, the contact between white wine and cork granulate causes acutissimin-A to be formed, and in this case in even greater quantities than those formed in the presence of red wine—the last column gives the average intensities of the signals, which are higher for the white wine.

### Example 4

For the purpose of perceiving the organoleptic changes caused by the contact of wine with cork, a wine tasting was arranged. A panel of 31 anonymous tasters and regular consumers of wine were given four samples of two wines, one white and one red, with the following characteristics:

Red wine; Year 2003; Grape variety—Cabernet Sauvignon & Touriga Nacional; Trademark—Calhandriz, Quinta da Romeira, Companhia das Quintas.

White wine; Year 2004; Grape variety—Moscatel Gráudo & Arinto; Trademark—Calhandriz, Quinta da Romeira, Companhia das Quintas.

Two out of the four samples corresponded to the wine “as is”, while the last two samples had been previously mixed with cork granulate (1.5 mm) in a proportion by volume of 15/100 (cork/wine) for 1 hour and filtered immediately before tasting.

The tasters were asked to rate the four samples according to three parameters: taste, aroma and colour, on a scale of 1 to 5, wherein 1 means weak and 5 means excellent. This was made as a blind assay. The gender and age of each one them also figured in the record sheet. The results are summarized in Table 4.

TABLE 4

| | | | RW1 - Red | | | RW2 - Red Wine | | | WW1 - White Wine | | | WW2 - White | | |
|---|---|---|---|---|---|---|---|---|---|---|---|---|---|---|
| | | | Wine As Is | | | Contact with Cork | | | Contact with Cork | | | Wine As Is | | |
| Taster | Gender | Age | T | A | C | T | A | C | T | A | C | T | A | C |
| 1 | F | 26 | 5 | 4 | 5 | 4 | 3 | 4 | 4 | 3 | 4 | 4 | 4 | 3 |
| 2 | M | 31 | 4 | 4 | 4 | 3 | 4 | 4 | 4 | 3 | 4 | 3 | 3 | 2 |
| 3 | M | 68 | 2 | 3 | 3 | 1 | 2 | 3 | 2 | 2 | 3 | 3 | 2 | 3 |
| 4 | M | 39 | 1 | 3 | 2 | 1 | 1 | 2 | 1 | 1 | 2 | 4 | 4 | 4 |
| 5 | F | 29 | 3 | 2 | 3 | 4 | 3 | 4 | 2 | 3 | 3 | 3 | 4 | 2 |
| 6 | F | 40 | 3 | 2 | 3 | 2 | 3 | 4 | 3 | 4 | 3 | 3 | 4 | 4 |
| 7 | F | 33 | 3 | 3 | 5 | 4 | 3 | 5 | 4 | 5 | 5 | 3 | 3 | 3 |
| 8 | M | 35 | 2 | 3 | 3 | 2 | 4 | 3 | 1 | 3 | 2 | 2 | 2 | 1 |
| 9 | F | 39 | 2 | 3 | 2 | 2 | 2 | 2 | 2 | 3 | 3 | 3 | 3 | 2 |
| 10 | F | 50 | 2 | 1 | 3 | 1 | 1 | 3 | 2 | 3 | 3 | 1 | 2 | 2 |
| 11 | F | 60 | 3 | 2 | 3 | 2 | 2 | 3 | 2 | 2 | 3 | 3 | 3 | 3 |
| 12 | F | 39 | 2 | 2 | 3 | 1 | 2 | 3 | 2 | 2 | 3 | 1 | 1 | 2 |
| 13 | M | 50 | 3 | 3 | 4 | 1 | 2 | 4 | 2 | 3 | 4 | 3 | 3 | 4 |
| 14 | F | 51 | 3 | 3 | 3 | 1 | 2 | 2 | 4 | 4 | 4 | 4 | 4 | 3 |
| 15 | F | 42 | 3 | 2 | 3 | 1 | 1 | 3 | 3 | 3 | 2 | 3 | 2 | 3 |
| 16 | M | 48 | 3 | 2 | 3 | 2 | 2 | 3 | 3 | 3 | 3 | 4 | 4 | 3 |
| 17 | M | 37 | 3 | 3 | 3 | 1 | 2 | 3 | 3 | 3 | 4 | 2 | 2 | 3 |
| 18 | M | 42 | 3 | 2 | 2 | 2 | 2 | 2 | 4 | 3 | 3 | 5 | 4 | 2 |
| 19 | M | 57 | 2 | 3 | 4 | 2 | 3 | 3 | 3 | 4 | 4 | 1 | 3 | 3 |
| 20 | M | 49 | 2 | 2 | 3 | 3 | 2 | 3 | 3 | 3 | 3 | 2 | 2 | 2 |
| 21 | F | 50 | 3 | 3 | 4 | 4 | 4 | 4 | 4 | 4 | 4 | 3 | 3 | 3 |
| 22 | F | 55 | 5 | 3 | 3 | 5 | 4 | 4 | 5 | 3 | 2 | 3 | 3 | 3 |
| 23 | M | 40 | 3 | 3 | 4 | 3 | 3 | 4 | 4 | 3 | 3 | 4 | 3 | 3 |
| 24 | M | 46 | 3 | 3 | 3 | 4 | 4 | 3 | 4 | 3 | 3 | 3 | 3 | 2 |
| 25 | F | 49 | 4 | 4 | 4 | 3 | 4 | 4 | 4 | 4 | 5 | 5 | 5 | 4 |
| 26 | M | 44 | 2 | 2 | 2 | 1 | 1 | 2 | 2 | 2 | 2 | 3 | 2 | 2 |
| 27 | M | 69 | 3 | 3 | 4 | 3 | 3 | 4 | 2 | 2 | 4 | 3 | 3 | 4 |
| 28 | F | 65 | 1 | 2 | 3 | 2 | 3 | 3 | 3 | 2 | 3 | 4 | 5 | 2 |
| 29 | F | 46 | 3 | 2 | 3 | 4 | 4 | 4 | 4 | 4 | 4 | 4 | 4 | 3 |
| 30 | M | 71 | 3 | 3 | 4 | 2 | 3 | 4 | 3 | 3 | 3 | 2 | 2 | 2 |
| 31 | F | 70 | 2 | 2 | 3 | 3 | 4 | 4 | 3 | 3 | 4 | 2 | 2 | 4 |

| | | | | | | | | | | | | | |
|---|---|---|---|---|---|---|---|---|---|---|---|---|---|
| Partial averages | 47.42 | 2.77 | 2.65 | 3.26 | 2.39 | 2.68 | 3.32 | 2.97 | 3.00 | 3.29 | 3.00 | 3.03 | 2.77 |

| | | | | |
|---|---|---|---|---|
| 3 parameter averages | 2.89 | 2.80 | 3.09 | 2.94 |

T—Taste; A—Aroma; C—Colour;

The shaded columns (RW2 and WW1) correspond to the wines which were in contact with cork, i.e. red wine and white wine respectively.

If we consider the average of the 3 parameters for each sample, we can generally conclude that the red wine remains almost unaltered and the white wine improves after being contacted with cork. The slight changes can be considered as falling within the natural variations associated with this kind of evaluation.

A more detailed analysis shows that in the case of the red wine, there is a rather significant decrease in the “Taste” parameter, while for the white wine there is a significant increase in the “Colour” parameter, meaning that the tasters gave importance to the colour of the wine which was put in contact with the cork. Both the red wine and the white wine showed an improved colour after contacting with the cork.

The change in the taste of the red wine was identified by the tasters as a “woody” touch which, although not being referred to as negative, was less appreciated by some of them. The treated white wine was frequently mentioned as being a rounder, less acid and “woody” wine. In general, it can be stated that the contact with cork did not significantly alter the red wine and, to a certain extent, it even enhanced the organoleptic properties of the white wine.

The wines used in this example were then analysed in order to detect the presence of acutissimin-A.

The results are presented in Table 5.

TABLE 5

| | Signal | Signal | | |
|---|---|---|---|---|
| | m/z = 915 | Intensity | Rt (signal | Average |
| SAMPLES | by EIC | m/z = 915 | of m/z = 915) | Intensities |
| White Wine_1 1^{st} INJ | Yes | 39 | 11.7 | 39.0 |
| White Wine_1 2^{nd} INJ | Yes | 39 | 11.7 | |
| White Wine_2 1^{st} INJ | No | — | — | — |
| White Wine_2 2^{nd} INJ | No | — | — | |
| Red Wine_1 1^{st} INJ | No | — | — | — |
| Red Wine_1 2^{nd} INJ | No | — | — | |
| Red Wine_2 1^{st} INJ | Yes | 10 | 11.6 | 8 |
| Red Wine_2 2^{nd} INJ | Yes | 6 | 11.6 | |

It may be concluded from the results presented in Table 5 that the wines which were put in contact with cork granulate have in their composition the compound acutissimin-A—last column of Table 5.

The above description includes all the information which is deemed necessary to enable persons skilled in the art to execute the claimed invention.

Although this invention has been described in detail with respect to its preferred embodiments, it is quite evident to those skilled in the art that several possible modifications can be made thereto without departing from the teachings of the invention. The scope and special characteristics of this invention are as defined in the attached claims.

### DESCRIPTION OF THE FIGURES

FIG. 1—Mass spectrum characteristic of acutissimin-A (sample of contact of red wine with cork granulate—30 minutes contact).

FIG. 2—Partial chromatogram of an as-is sample of red wine which was in contact with cork granulate for 30 minutes.

FIG. 3—Partial chromatogram of an as-is sample of red wine which was in contact with cork granulate for 150 minutes.

FIG. 4—Partial chromatogram of an as-is sample of red wine which was in contact with cork granulate for 6 days.

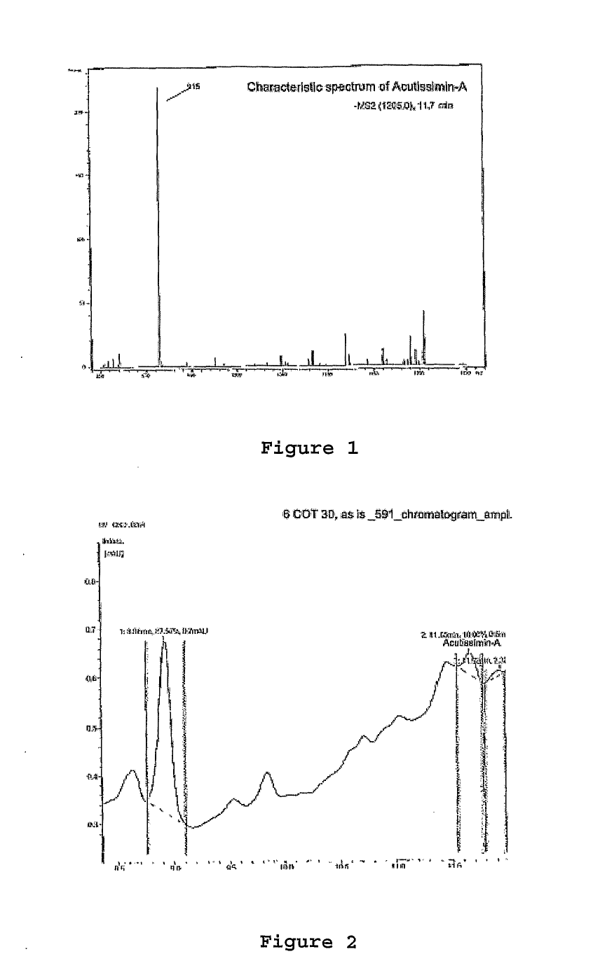

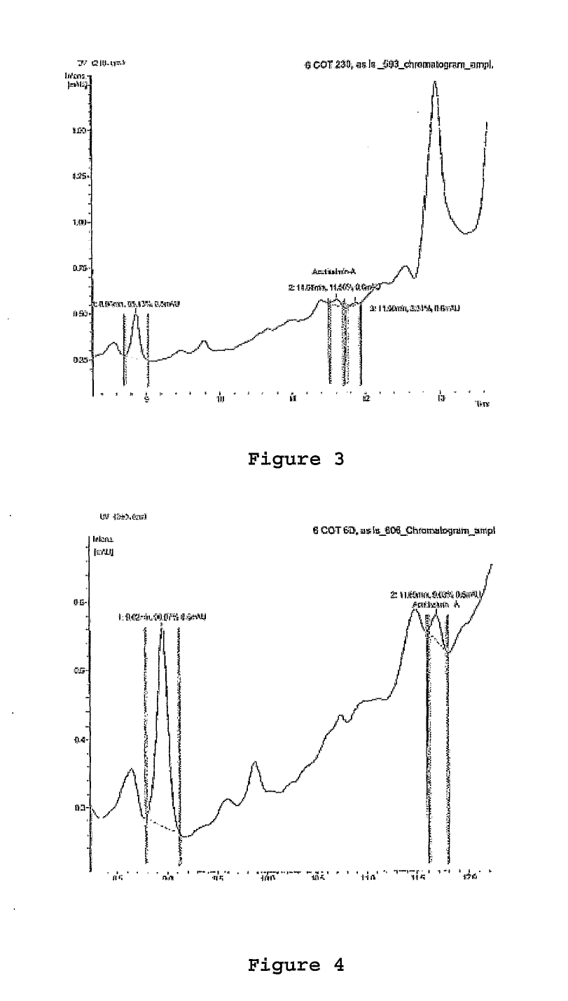

1. A process for the additivation of wine based on the contact thereof with natural materials other than oak wood, namely cork, under conditions where certain compounds of the said natural materials, namely vescalagin, react with other compounds present in the wine, characterized in that an anti-tumour compound named acutissimin-A forms in the wine, the said contact being susceptible of occurring in different situations and by various methods, at room temperature and over a variable period of contact.

2. A process according to claim 1, characterized in that the material used contains vescalagin.

3. A process according to claim 1, characterized in that the material used is cork from Quercus suber L.

4. A process according to claim 1, characterized in that the contact between the natural materials and the wine is made in one or more of three situations:
a. At the final stage of vinification, before bottling
b. During bottling
c. Immediately before consumption

5. A process according to claim 1, characterized in that the cork is used in finely divided particles or in the form of chips.

6. A process according to claim 1, characterized in that the natural materials can be added to the wine as they are and be separated at a later stage by filtration.

7. A process according to claim 1, characterized in that the natural materials can be added to the wine inside retention systems which contain the cork and can be easily introduced into and removed from the wine containers.

8. A process according to claim 7, characterized in that said retention systems are porous paper cartridges, textile material cartridges, porous capsules, perforated metal frameworks or net systems and other similar systems.

9. A process according to claim 1, characterized in that the contact between the natural materials and the wine occurs preferably at a temperature range from 5 to 30° C., over a period of contact from about 10 minutes to several months, and with cork having a granulometry from 100 μm to 30 mm or in the form of chips.

10. A process according to claim 10, characterized in that the preferred conditions of contact are at room temperature, for at least 30 minutes, with a cork granulometry of 1 mm.

11. A process according to claim 1, characterized in that the cork particles used in these systems are optionally sterilized, before or after their closure into the systems, preferably by gamma ray or electron beam irradiation.

12. A process according to claim 1, characterized in that there is also an organoleptic change in the wine.
